(19) United States
(12) Patent Application Publication
(10) Pub. No.: US 2009/0093851 A1
(43) Pub. Date: Apr. 9, 2009

(54) Transfacet-Pedicle Locking Screw Fixation of Lumbar Motion Segment

(76) Inventor: Said G. Osman, Russellville, AL (US)

Correspondence Address: NIXON & VANDERHYE, PC, 901 NORTH GLEBE ROAD, 11TH FLOOR, ARLINGTON, VA, 22203, US

(21) Appl. No.: 12/248,154

(22) Filed: Oct. 9, 2008

Related U.S. Application Data
(60) Provisional application No. 60/978,596, filed on Oct. 9, 2007.

(51) Int. Cl. A61B 17/04

(52) U.S. Cl. 606/301; 606/304; 606/305

(57) ABSTRACT

A transfacet-pedicle locking screw fixation assembly includes a plurality of pedicle screws each including threads at a distal end and a connecting receptacle at a proximal end. The connecting receptacle is oriented perpendicularly with respect to a longitudinal axis of the pedicle screw. A basic transfacet locking screw including threads at least at a distal end has the distal end engaging the connecting receptacle in one of the pedicle screws. The transfacet locking screw may also be interlocked with another transfacet locking screw.

### CROSS-REFERENCES TO RELATED APPLICATIONS

This application claims the benefit of U.S. Provisional Patent Application Ser. No. 60/978,596, filed Oct. 9, 2007, the entire content of which is herein incorporated by reference.

### STATEMENT REGARDING FEDERALLY SPONSORED RESEARCH OR DEVELOPMENT

(NOT APPLICABLE)

### BACKGROUND OF THE INVENTION

The invention relates to apparatus and methods for spinal fixation and, more particularly, to a transfacet-pedicle locking screw fixation device and method of insertion for stabilizing a facet joint and an adjacent target pedicle.

Instrumented spinal fixation is in common practice. Pedicle screw fixation requires instrumentation of the pedicles on either side of the target disc. Even with so-called minimally invasive pedicle screw fixation, there is significant surgical trauma, and clinically significant hardware impingement on soft tissues since the locations of the screw heads and the rods are inter/intramuscular. Less invasive and less bulky fixation approaches are the various versions of transfacet fixation. The problem with the prior facet fixation, however, is the inherent inability of the construct to withstand the physiological torque experienced by the lumbar motion segments. Some designs of the facet fixation involve trans-spinous process screw implantation. While this may give a good support on the side of the inferior facet, it does not affect the strength of the fixation on the superior facet side.

### BRIEF SUMMARY OF THE INVENTION

The transfacet-pedicle locking screw fixation device and method of the invention overcomes the drawbacks of the prior art and achieves marked improvement over the prior art. In particular, the invention provides the least invasive of all current lumbar spine fixation devices since only the pair of facet joint and only the pair of pedicles (of the same vertebra) are instrumented, hence reducing the risk of injury to the cephalad facet joint (as is the case with pedicle screw instrumentation) by 100%. Since the fixation is entirely intra-osseous and transfacet, there is minimal risk for muscle irritation. Moreover, there is hardly any recordable blood loss since the procedure is entirely percutaneous. Additionally, due to less hardware implantation required and minimal wound closure, the operative time is significantly reduced.

The assembly of the described embodiments combines the positive attributes of the pedicle screws and transfacet fixation, that is, minimal surgical trauma and a strong reliable fixation. It markedly reduces the patient's need for pain medication, and it markedly reduces patient down time. Combined with the technique of least invasive interbody fusion, it will provide a 360 degree spinal fusion whose stability will challenge any construct currently available for clinical use, while maintaining the least surgical trauma and minimal blood loss.

In an exemplary embodiment, a transfacet-pedicle locking screw fixation assembly includes a pedicle screw with threads at a distal end and a connecting receptacle at a proximal end. The assembly additionally includes at least one of (1) a basic transfacet locking screw including threads at least at a distal end. The transfacet locking screw is engageable with the connecting receptacle in the pedicle screw. (2) A female transfacet locking screw includes threads at least at a distal end and a threaded opening at proximal end. The distal end of the female transfacet locking screw is engageable with the connecting receptacle in the pedicle screw. (3) A male transfacet locking screw includes threads at least at a distal end and at a proximal end. The threads at the proximal end are engageable with the threaded opening in the female transfacet locking screw, and the distal end of the male transfacet locking screw is engageable with the connecting receptacle in the pedicle screw. Preferably, the pedicle screw, the basic transfacet locking screw, the female transfacet locking screw and the male transfacet lacking screw are cannulated.

The connecting receptacle of the pedicle screw may include a threaded opening oriented perpendicularly to a longitudinal axis of the pedicle screw. Alternatively, the connecting receptacle of the pedicle screw may include an opening oriented perpendicularly to a longitudinal axis of the pedicle screw and a threaded channel oriented in alignment with the longitudinal axis and cooperable with the opening. The transfacet-pedicle locking screw fixation assembly may additionally include a set screw affixable in the threaded channel.

The transfacet-pedicle locking screw fixation assembly may additionally include a cannulated locking pedicle screw driver that includes a shaft that is positionable in alignment with a longitudinal axis of the pedicle screw and at least a first wire guide disposed perpendicularly and attached to the shaft. In this context, the locking pedicle screw driver may include a second wire guide attached to the first wire guide.

Preferably, the pedicle screw, the basic transfacet locking screw, the female transfacet locking screw and the male transfacet lacking screw are formed of at least one of a metal alloy, a bioabsorbable material, or a bio-incorporable material.

In another exemplary embodiment, a transfacet-pedicle locking screw fixation assembly includes a plurality of pedicle screws each including threads at a distal end and a connecting receptacle at a proximal end. The connecting receptacle is oriented perpendicularly with respect to a longitudinal axis of the pedicle screw. A basic transfacet locking screw including threads at least at a distal end has the distal end engaging the connecting receptacle in one of the pedicle screws.

In still another exemplary embodiment, a method of implanting a transfacet-pedicle locking screw fixation assembly for stabilizing a patient facet joint and an adjacent target pedicle includes the steps of (a) inserting a pedicle screw into the target pedicle, the pedicle screw including threads at a distal end and a connecting receptacle at a proximal end; (b) securing the pedicle screw with a pedicle screw driver and orienting the pedicle screw with the connecting receptacle facing the facet joint; (c) inserting a transfacet locking screw through the patient spinous process, the patient lamina, and the facet joint, across the pedicle medial wall and into the connecting receptacle of the pedicle screw; and (d) securing the transfacet locking screw in the connecting receptacle.

Step (c) may be practiced by (c-1) attaching a locking screw wire guide to a shaft of the pedicle screw driver; (c-2) passing a locking screw guide wire through the locking screw wire guide, through the patient skin to a base of the spinous process; (c-3) driving the locking screw guide wire through the spinous process, the lamina, and the facet joint, across the pedicle medial wall and into the connecting receptacle of the pedicle screw; and (c-4) inserting the transfacet locking screw over the locking screw guide wire into the connecting receptacle of the pedicle screw.

In yet another exemplary embodiment, a method of implanting a transfacet-pedicle locking screw fixation assembly for stabilizing a patient facet joint and an adjacent target pedicle includes the steps of (a) inserting a first pedicle screw into the target pedicle, the first pedicle screw including threads at a distal end and a first connecting receptacle at a proximal end; (b) securing the first pedicle screw with a first pedicle screw driver and orienting the first pedicle screw with the first connecting receptacle facing the facet joint; (c) inserting a second pedicle screw into a contralateral pedicle, the second pedicle screw including threads at a distal end and a second connecting receptacle at a proximal end; (d) securing the second pedicle screw with a second pedicle screw driver; (e) inserting a female transfacet locking screw into engagement with the second connecting receptacle, the female transfacet locking screw including threads at least at a distal end and a threaded opening at a proximal end, the inserting step comprising orienting the threaded opening to be perpendicular to an access path for a male transfacet locking screw; and (f) inserting the male transfacet locking screw along the access path and securing the male transfacet locking screw in the threaded opening and the first connecting receptacle.

### BRIEF DESCRIPTION OF THE DRAWINGS

These and other aspects and advantages will be described in detail with reference to the accompanying drawings, in which:

FIG. 1 shows an exemplary basic transfacet locking screw;

FIG. 1A shows an exemplary socket for the transfacet locking screws;

FIG. 2 shows an exemplary female transfacet locking screw;

FIG. 3 illustrates a locking pedicle screw;

FIG. 4 illustrates the locking pedicle screw including a set screw for securing the transfacet locking screw;

FIG. 5 illustrates an exemplary male transfacet locking screw;

FIG. 6 shows the pedicle screw driver and wire guide assembly; and

FIG. 7 shows the transfacet-pedicle locking screw fixation assembly secured in a vertebral space.

### DETAILED DESCRIPTION OF THE INVENTION

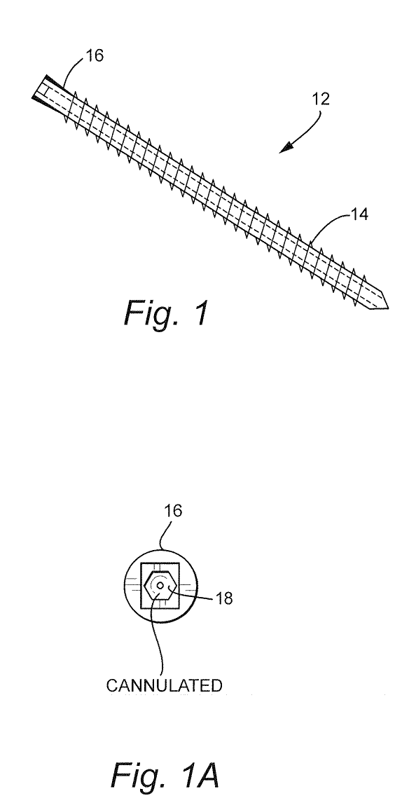

With reference to the drawings, a transfacet-pedicle locking screw fixation assembly includes a plurality of components that provide for spine fixation using only one pair of facet joints and only one pair of pedicles (of the same vertebra), thereby reducing the risk of injury to the cephalad facet joint. The assembly includes a first male transfacet locking screw 12 including threads 14 at least at a distal end. Preferably, the locking screw 12 is fully threaded and cannulated. The locking screw 12 can be made of a metal alloy, a bioabsorbable material, or a bio-incorporable material or other suitable material of variable lengths and diameters. The length of the screw 12 varies according to the patient's anatomy. As shown in FIG. 1A, the screw head 16 includes a shaped socket 18 that is shaped to receive a suitable screw driver (discussed in more detail below). As shown, the socket 18 is preferably hexagonal shaped, although other shapes may be suitable.

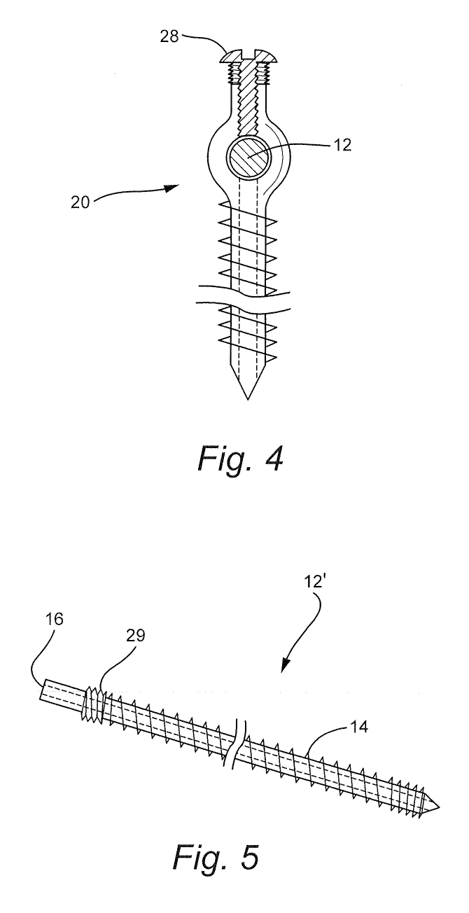

With reference to FIGS. 3 and 4, assembly preferably includes one or more locking pedicle screws 20 that are preferably cannulated for percutaneous insertion. The locking pedicle screws 20 are similarly formed of a metal alloy, a bioabsorbable material, or a bio-incorporable material or other suitable material and are of variable lengths and diameters. As shown, the locking pedicle screws 20 may be fully threaded with threads 22 deep enough to get traction into the cancellous bone. Near the proximal end of the screw 20 is a connecting receptacle 24 for receiving the transfacet locking screw 12. The connecting receptacle 24 may be a threaded hole 25 for receiving the locking screw 12 in a threaded connection or may include a non-threaded hole 25 oriented perpendicularly to a longitudinal axis of the pedicle screw 20 and cooperable with a threaded channel 26 oriented in alignment with the longitudinal axis of the pedicle screw 20. With the transfacet locking screw 12 inserted in the hole 25, a set screw 28 is securable in the threaded channel 26 to lock the transfacet locking screw 12 to the pedicle screw 20.

In one embodiment, the locking screw 12 is inserted from one side across the spinous process and the lamina and across the contralateral facet joint. The distal end of the locking screw 12 fits into the pedicle screw opening 25 of the ipsilateral pedicle (on the side of the fixed facet joint). The locking screw 12 may be locked into the pedicle screw hole 25 by a threaded connection or by use of the set screw 28. The proximal end of the screw 12 is embedded in the lamina adjoining the fixed facet joint. When fully seated in the pedicle screw 20, the locking screw 12 will have completely traversed the spinous process, and no part of it is left seated in the spinous process. In another embodiment, the locking screw 12 does not traverse the spinous process but only the ipsilateral lamina and facet joint.

FIG. 5 illustrates a male transfacet locking screw 12′ that forms part of the fixation assembly. The locking screw 12′ preferably includes threads 14 at least at a distal end but is preferably fully threaded as shown. The male transfacet locking screw 12′ additionally includes proximal threads 29 engageable with an opening in a female transfacet locking screw 30 (discussed below). The locking screw 12′ is cannulated and formed of a metal alloy, a bioabsorbable material, or a bio-incorporable material or other suitable material of variable lengths and diameters. The length of the screw 12′ will vary according to the anatomy of the patient. In one embodiment, the screw 12′ is inserted from one side across the spinous process and the lamina and across the contralateral facet joint. The distal end of the locking screw 12′ fits into the pedicle screw opening 25 of the ipsilateral pedicle. Like locking screw 12, the locking screw 12′ may be locked into the pedicle screw opening 25 by a threaded connection or by use of the set screw 28. The proximal end of the locking screw 12′ is also preferably threaded and locks into the near end of the contralateral female locking screw 30. In another embodiment, the locking screw 12′ does not traverse the female locking screw 30 to the contralateral facet joint, but lies freely in the spinous process.

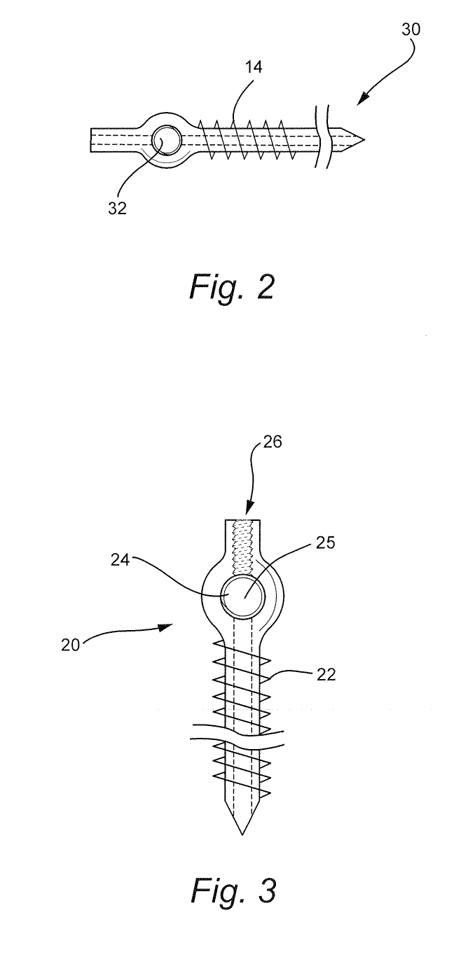

FIG. 2 shows the female locking screw 30 of the fixation assembly. The female locking screw 30 is preferably fully threaded but includes threads 14 at least at a distal end. A threaded opening 32 is provided at a proximal end that receives the proximal end of male locking screw 12′ via threads 29 for the contralateral transfacet fixation. The distal end of the female locking screw 30 penetrates the pedicle screw 20 and may be locked in the pedicle screw hole 25 by a threaded connection or the set screw 28.

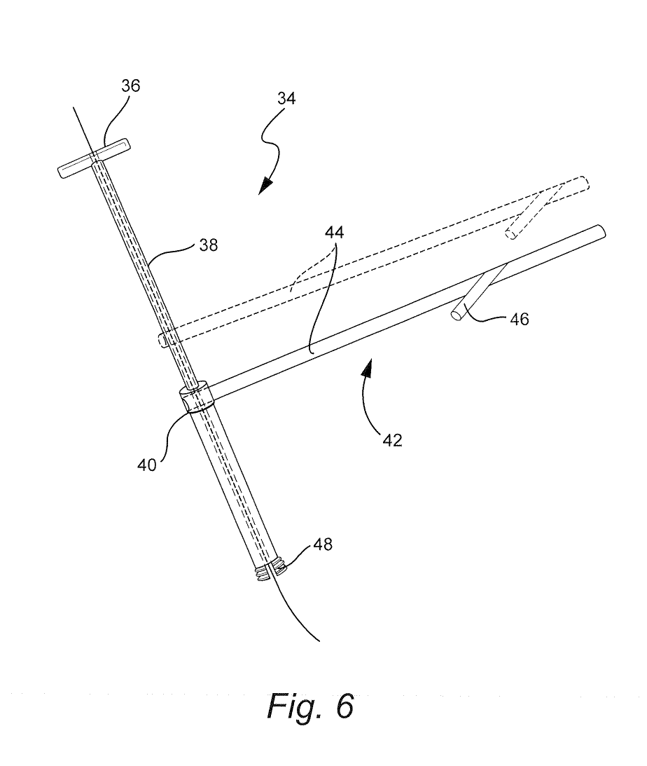

FIG. 6 illustrates the locking pedicle screw driver 34 that serves to lock the pedicle screws 20 and also to support wire guides for the locking screws of the fixation assembly. The screwdriver 34 is cannulated for the insertion of the locking pedicle screw 20. It is threaded over a previously-inserted guide wire into the pedicle. The driver has a T-shaped handle 36 that is oriented parallel to the locking screw hole 25 in the pedicle screw 20. This orientation helps the surgeon determine the orientation of the screw head in the pedicle. The shaft 38 of the pedicle screwdriver 34 has a shoulder or notch 40 on which a locking screw wire guide 42 rests or locks. The shaft 38 may include multiple notches 40 to accommodate different volumes of the soft tissues to be traversed by the instrument. FIG. 6 shows alternative positions of the wire guide 42 on the pedicle screwdriver shaft 38 depending on the depth of the vertebra.

The transfacet locking screw wire guide 42 includes a horizontal arm 44 that bears on the guides of the ipsilateral locking screw. A wire guide 46 is oriented such that when the guide wire is passed through it, the wire will pass through the hole 25 in the pedicle screw 20. The wire is preferably made of a reasonably stiff alloy to avoid deflection by minor obstacles.

A cannulated drill or the like is used to drill the path of the locking screws through the spinous process, the lamina, the facets and the medial wall of the pedicle.

Like the pedicle screwdriver, a transfacet screwdriver is a cannulated screwdriver that is threaded over the guide wire to facilitate insertion of the locking screws. The screwdriver is preferably provided with a shaped tip 48 that fits into the shaped recess 18 of the locking screw head 16. The female transfacet locking screw 30 preferably has a rectangular head with its wider dimension being parallel with the hole 32 for receiving the male locking screw 12, 12′ from the contralateral side. The wide dimension of the driver tip 48 and the hole in the female screwdriver are in turn parallel to the T-shape of the driver T-shaped handle 36.

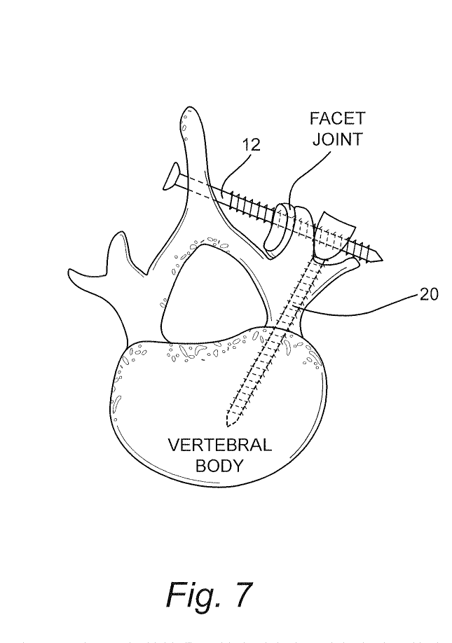

An exemplary surgical technique for the implantation of the transfacet locking screw fixation assembly will be described. The patient is first placed in a prone position on a radiolucent operating table. Under fluoroscopic control, the level of the target motion segment is marked on the skin. The assembly of trocar and cannula are inserted into the target pedicle under fluoroscopic control. The trocar is removed, and the guide wire is introduced into the pedicle and the vertebral body through the cannula. The cannula is removed while holding the guide wire in place. Tapping of the pedicle canal is carried out with a tap of appropriate diameter. The appropriate sized pedicle screw is inserted over the guide wire to the appropriate depth so that the transfacet screw will be able to fit into it. The screw should be seated such that the locking screw hole faces the facet joint. The locking screw wire guide is attached to the pedicle screwdriver at the appropriate level depending on the size of the patient. The locking screw guide wire is passed through the guide, through the skin to the base of the spinous process. From this point, it is driven with the power drill through the spinous process, the lamina, the facet joint and across the medial wall of the pedicle into the pedicle screw. While protecting the soft tissue with a sleeve, the locking screw drill is inserted over the guide wire into the base of the spinous process, the lamina, the facet joint and the pedicle. Tapping is carried out and an appropriate length locking screw is inserted. This may be self tapping in which case the tapping step is omitted. During the locking screw insertion, the guide wire in the pedicle screw is pulled out enough to allow passage of the locking screw. The pedicle set screw is inserted over the guide wire to lock the locking screw in place. A similar procedure is repeated the contralateral side. The implanted fixation assembly is illustrated in FIG. 7.

In the implantation of interlocking transfacet locking screws, the same steps are used up to the insertion of the transfacet locking screw. The female locking screw is inserted so that the hole for the male counterpart is perpendicular to the path of the contralateral male locking screw, as shown by the orientation of the T-handle of the screwdriver. The locking screw guide is left in place and the steps of the contralateral pedicle screw insertion are carried out. The bar with double locking screw wire guide is placed over the second pedicle screwdriver shaft as previously described and used with the ipsilateral guide transmitting the previously placed locking screw wire. The new wire is then placed in the contralateral guide and driven through the hole in the female locking screw, lamina, facet joint and pedicle screw as previously described. Drilling of the screw path is carried out as previously described. The male locking screw is now inserted into the prepared track and locked into both the female locking screw and the pedicle screw. The pedicle set screw is used to lock the locking screw as previously described. All the instruments are removed after implantation.

With the fixation assembly and methodology described, transfacet-pedicle locking screw fixation of a lumbar motion segment is the least invasive of all current lumbar spine fixation devices since only one pair of facet joints and only one pair of pedicles (of the same vertebra) are instrumented, thereby reducing the risk of injury to the cephalad facet joint. Since the fixation is entirely intra-osseous and transfacet, there is minimal risk for muscle irritation. Moreover, there is hardly any recordable blood loss since the procedure is entirely percutaneous. Additionally, due to less hardware implantation required and minimal wound closure, the operative time is significantly reduced.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A transfacet-pedicle locking screw fixation assembly comprising:
a pedicle screw including threads at a distal end and a connecting receptacle at a proximal end; and
at least one of: (1) a basic transfacet locking screw including threads at least at a distal end, the transfacet locking screw being engageable with the connecting receptacle in the pedicle screw, (2) a female transfacet locking screw including threads at least at a distal end and a threaded opening at proximal end, the distal end of the female transfacet locking screw being engageable with the connecting receptacle in the pedicle screw, and (3) a male transfacet locking screw including threads at least at a distal end and at a proximal end, the threads at the proximal end being engageable with the threaded opening in the female transfacet locking screw, and the distal end of the male transfacet locking screw being engageable with the connecting receptacle in the pedicle screw.

2. A transfacet-pedicle locking screw fixation assembly according to claim 1, wherein the pedicle screw is cannulated.

3. A transfacet-pedicle locking screw fixation assembly according to claim 1, wherein the basic transfacet locking screw, the female transfacet locking screw and the male transfacet lacking screw are cannulated.

4. A transfacet-pedicle locking screw fixation assembly according to claim 1, wherein the connecting receptacle of the pedicle screw comprises a threaded opening oriented perpendicularly to a longitudinal axis of the pedicle screw.

5. A transfacet-pedicle locking screw fixation assembly according to claim 1, wherein the connecting receptacle of the pedicle screw comprises an opening oriented perpendicularly to a longitudinal axis of the pedicle screw and a threaded channel oriented in alignment with the longitudinal axis and cooperable with the opening, the transfacet-pedicle locking screw fixation assembly further comprising a set screw affixable in the threaded channel.

6. A transfacet-pedicle locking screw fixation assembly according to claim 1, further comprising a cannulated locking pedicle screw driver, the locking pedicle screw driver including a shaft that is positionable in alignment with a longitudinal axis of the pedicle screw and at least a first wire guide disposed perpendicularly and attached to the shaft.

7. A transfacet-pedicle locking screw fixation assembly according to claim 6, wherein the locking pedicle screw driver comprises a second wire guide attached to the first wire guide.

8. A transfacet-pedicle locking screw fixation assembly according to claim 1, wherein the pedicle screw, the basic transfacet locking screw, the female transfacet locking screw and the male transfacet lacking screw are formed of at least one of a metal alloy, a bioabsorbable material, or a bio-incorporable material.

9. A transfacet-pedicle locking screw fixation assembly comprising:
a plurality of pedicle screws each including threads at a distal end and a connecting receptacle at a proximal end, the connecting receptacle being oriented perpendicularly with respect to a longitudinal axis of the pedicle screw; and
a basic transfacet locking screw including threads at least at a distal end, the distal end engaging the connecting receptacle in one of the pedicle screws.

10. A transfacet-pedicle locking screw fixation assembly according to claim 9, further comprising a female transfacet locking screw including threads at least at a distal end and a threaded opening at a proximal end, the distal end of the female transfacet locking screw engaging the connecting receptacle in one of the pedicle screws.

11. A transfacet-pedicle locking screw fixation assembly according to claim 9, further comprising a male transfacet locking screw including threads at least at a distal end and at a proximal end, the threads at the proximal end engaging the threaded opening in the female transfacet locking screw, and the distal end of the male transfacet locking screw engaging the connecting receptacle in one of the pedicle screws.

12. A method of implanting a transfacet-pedicle locking screw fixation assembly for stabilizing a patient facet joint and an adjacent target pedicle, the method comprising:
(a) inserting a pedicle screw into the target pedicle, the pedicle screw including threads at a distal end and a connecting receptacle at a proximal end;
(b) securing the pedicle screw with a pedicle screw driver and orienting the pedicle screw with the connecting receptacle facing the facet joint;
(c) inserting a transfacet locking screw through the patient spinous process, the patient lamina, and the facet joint, across the pedicle medial wall and into the connecting receptacle of the pedicle screw; and
(d) securing the transfacet locking screw in the connecting receptacle.

13. A method according to claim 12, wherein step (c) is practiced by:
(c-1) attaching a locking screw wire guide to a shaft of the pedicle screw driver;
(c-2) passing a locking screw guide wire through the locking screw wire guide, through the patient skin to a base of the spinous process;
(c-3) driving the locking screw guide wire through the spinous process, the lamina, and the facet joint, across the pedicle medial wall and into the connecting receptacle of the pedicle screw; and
(c-4) inserting the transfacet locking screw over the locking screw guide wire into the connecting receptacle of the pedicle screw.

14. A method according to claim 12, wherein the connecting receptacle is a threaded opening, and wherein step (d) is practiced by threading the transfacet locking screw into the threaded opening.

15. A method according to claim 12, wherein the connecting receptacle of the pedicle screw is an opening oriented perpendicularly to a longitudinal axis of the pedicle screw and includes a threaded channel oriented in alignment with the longitudinal axis and cooperable with the opening, and wherein step (d) is practiced by securing a set screw in the threaded channel in engagement with the transfacet locking screw.

16. A method according to claim 12, further comprising interlocking the transfacet locking screw with another transfacet locking screw.

17. A method of implanting a transfacet-pedicle locking screw fixation assembly for stabilizing a patient facet joint and an adjacent target pedicle, the method comprising:
(a) inserting a first pedicle screw into the target pedicle, the first pedicle screw including threads at a distal end and a first connecting receptacle at a proximal end;
(b) securing the first pedicle screw with a first pedicle screw driver and orienting the first pedicle screw with the first connecting receptacle facing the facet joint;
(c) inserting a second pedicle screw into a contralateral pedicle, the second pedicle screw including threads at a distal end and a second connecting receptacle at a proximal end;
(d) securing the second pedicle screw with a second pedicle screw driver;
(e) inserting a female transfacet locking screw into engagement with the second connecting receptacle, the female transfacet locking screw including threads at least at a distal end and a threaded opening at a proximal end, the inserting step comprising orienting the threaded opening to be perpendicular to an access path for a male transfacet locking screw; and
(f) inserting the male transfacet locking screw along the access path and securing the male transfacet locking screw in the threaded opening and the first connecting receptacle.
